(19) United States
(12) Patent Application Publication
(10) Pub. No.: US 2007/0282290 A1
(43) Pub. Date: Dec. 6, 2007

(54) Absorbent article having a channeled absorbent layer and method of making the same

(76) Inventors: Robert Cole, Jackson, NJ (US); Audra Niszczak, Southampton, PA (US); Linda Morrell-Schwartz, Bensalem, PA (US)

Correspondence Address: RATNERPRESTIA, P O BOX 980, VALLEY FORGE, PA, 19482-0980, US

(21) Appl. No.: 11/890,566

(22) Filed: Aug. 7, 2007

Related U.S. Application Data
(62) Division of application No. 11/077,055, filed on Mar. 10, 2005.

(51) Int. Cl. A61F 13/532

(52) U.S. Cl. 604/385.101

(57) ABSTRACT

This invention provides an absorbent article having an absorbent layer including elongated longitudinal portions which thereby define an elongated gap. The elongated gap is defined by lateral spacing of the elongated longitudinal portions of the absorbent layer from one another. The invention is also directed to the method of manufacturing the absorbent article and the method of forming the elongated gap within the absorbent layer.

### CROSS REFERENCE TO RELATED APPLICATIONS

This application is a divisional application of U.S. patent application Ser. No. 11/077,055, filed Mar. 10, 2005, which is incorporated herein by reference in its entirety.

### FIELD OF THE INVENTION

This invention relates to absorbent articles. More particularly, it relates to absorbent articles having an absorbent layer defining a channel or gap.

### BACKGROUND OF THE INVENTION

Absorbent articles such as disposable diapers, training pants, adult incontinence garments, feminine hygiene pads and the like are known, their major function being to absorb and contain body exudates. Such articles are thus intended to prevent the soiling, wetting, or other contamination of clothing or other articles, such as bedding, that come into contact with the wearer. In the case of disposable diapers or feminine hygiene pads, for example, they are optionally provided with a basic structure that includes a liquid permeable cover, a liquid impermeable backsheet, an absorbent layer positioned between the cover and the backsheet, and a transfer layer for distributing the liquid more uniformly over the absorbent layer, positioned between the cover and the absorbent layer.

For background purposes, FIG. 1 shows a cross-sectional end view of a conventional absorbent article 100, which generally includes a cover 110, a transfer layer 120, a barrier layer 140 and an absorbent layer 130. Cover 110 is a liquid permeable layer allowing the passage of a liquid insult to transfer layer 120. Transfer layer 120 is configured to allow the liquid to be distributed more uniformly from the initial point of insult to the remainder of transfer layer 120. Absorbent layer 130 absorbs the liquid while barrier layer 140 prevents leakage of unabsorbed liquid. The article also has a positioning adhesive layer 141 to secure the absorbent article in place, such as to an undergarment of the wearer of the absorbent article. The adhesive layer is covered with releasable paper 142. Cover 110 and backsheet 140 are sealed together at location 143 to seal the absorbent article together.

While many developments have been made in the art of absorbent articles to improve performance, there remains a need for further performance improvements.

### SUMMARY OF THE INVENTION

In one aspect, the invention provides an absorbent article including a barrier layer configured to prevent the passage of liquid, a cover, and an absorbent layer interposed between the barrier layer and the cover. The absorbent layer has elongated portions that are laterally spaced from one another, thereby defining a gap between the laterally spaced portions of the absorbent layer.

In another aspect, the invention provides an absorbent article including a barrier layer, a cover, and an absorbent layer interposed between the cover and the barrier layer. The absorbent layer is configured to absorb an initial insult at an initial absorbency rate and to further absorb a subsequent insult at a subsequent absorbency rate faster than the initial absorbency rate.

In a further aspect, the invention provides a method of forming an absorbent article. The method includes interposing an absorbent layer having elongated portions that are laterally spaced from one another between a cover layer and a barrier layer. The laterally spaced portions define an elongated gap between the laterally spaced portions of the absorbent layer.

### BRIEF DESCRIPTION OF THE DRAWINGS

The invention is best understood from the following detailed description when read in connection with the accompanying drawing. It is emphasized that, according to common practice, the various features of the drawing are not to scale. On the contrary, the dimensions of the various features are arbitrarily expanded or reduced for clarity. Included in the drawing are the following figures:

FIG. 1 is a schematic cross-sectional end view of a conventional absorbent article;

FIG. 2A is a schematic end view of an exemplary embodiment of an absorbent article according to one aspect of this invention, shown during a pre-absorption stage (i.e., before the introduction of a liquid insult).

FIG. 2B is a schematic end view of the absorbent article illustrated in FIG. 2A, shown during a post-absorption stage (i.e., after the introduction of a liquid insult).

FIG. 3A is a schematic end view of another exemplary embodiment of an absorbent article according to one aspect of this invention, shown during a pre-absorption stage.

FIG. 3B is a schematic end view of the absorbent article illustrated in FIG. 3A, shown during a post-absorption stage.

FIG. 4 is a top view illustration of yet another exemplary embodiment of an absorbent article according to an aspect of this invention.

FIG. 5 is a top view illustration of still another exemplary embodiment of an absorbent article according to an aspect of this invention.

FIG. 6A is a top view illustration of another embodiment of an absorbent article according to an aspect of this invention.

FIG. 6B is a schematic cross-sectional end view of the absorbent article illustrated in FIG. 6A.

FIGS. 7A, 7B and 7C are schematic sided views illustrating an exemplary method of making absorbent layer portions according to an aspect of this invention.

FIG. 8 is a flow diagram illustrating an exemplary method of making an absorbent article.

FIG. 9A is a perspective illustration of a modified strike-through plate (Absorbency Rate Tester) that can be used to test an absorbent article according to an aspect of this invention.

FIG. 9B is a front view of the Absorbency Rate Tester shown in FIG. 9A.

FIGS. 9C and 9D are top and side views, respectively, of a top plate component of the Absorbency Rate Tester shown in FIG. 9B.

FIGS. 9E and 9F are top and detail views, respectively, of a bottom plate component of the Absorbency Rate Tester shown in FIG. 9B.

FIGS. 9G and 9H are top and side views, respectively, of a body component of the Absorbency Rate Tester shown in FIG. 9B.

FIGS. 9I, 9J and 9K are top, side, and cross-sectional side views, respectively, of a plate component of the Absorbency Rate Tester shown in FIG. 9B.

FIG. 10 is an illustration of a weight that can be used to test an absorbent article according to an aspect of this invention.

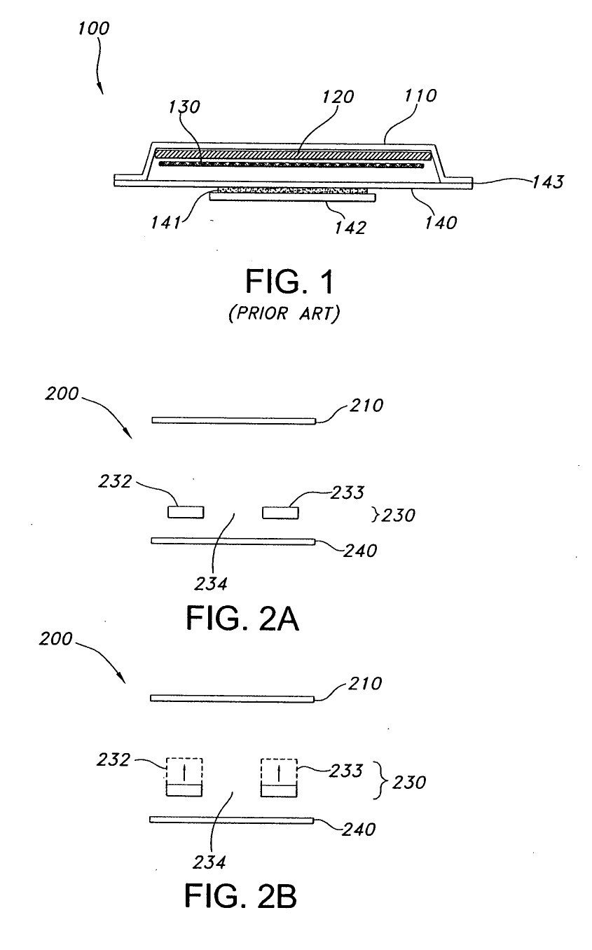

### DETAILED DESCRIPTION OF THE INVENTION

The invention is best understood from the following detailed description when read in connection with the accompanying drawing, which shows exemplary embodiments of the invention selected for illustrative purposes. The invention will be illustrated with reference to the Figures. Such Figures are intended to be illustrative rather than limiting and are included herewith to facilitate the explanation of the present invention.

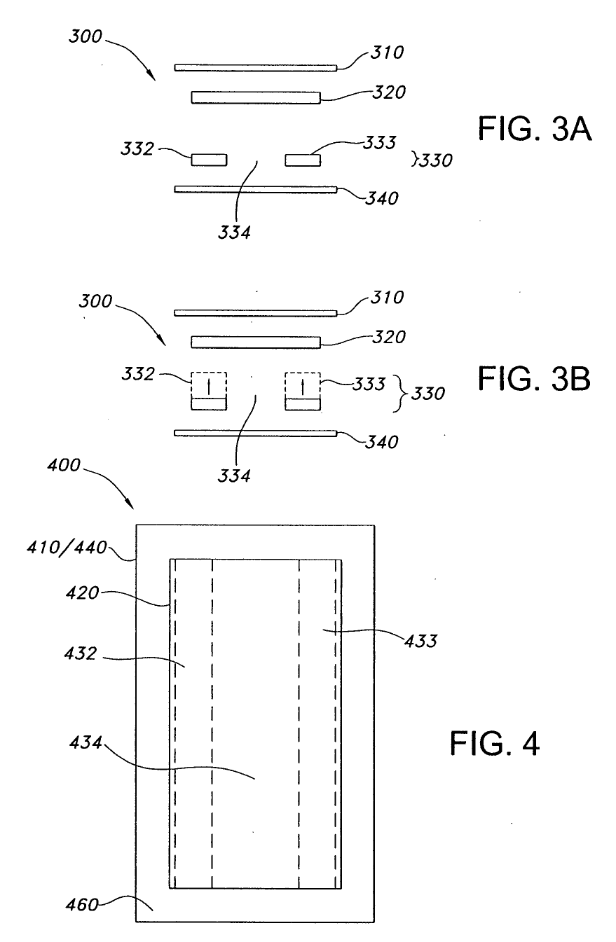

Referring generally to the drawing (specifically, FIGS. 2-7), illustrated embodiments of the present invention provide an absorbent article with an improved absorbent layer. With reference to FIGS. 2A and 2B, an absorbent article 200 is provided with a cover 210, a barrier layer 240, and an absorbent layer 230 interposed therebetween. Cover 210 may provide a transfer layer, a topsheet, or a transfer layer and topsheet combined.

Generally, a transfer layer is a fluid distribution layer and may be positioned adjacent and coextensive with the absorbent layer, on the side of the absorbent layer nearer the wearer, to improve distribution of bodily fluid more evenly over the full width and length of the absorbent layer. Such a layer serves to manage, transport, accommodate and/or direct high volumes and high flow rates of urine or other bodily fluids into the absorbent layer.

Cover 210 can be made from any of a number of materials known in the art, including for example, fibrous materials. The cover layer may be made from a nonwoven material, which may be thermoplastic fibers or filaments, for example. Shape-retaining nonwoven fabrics are well known and are made by a variety of processes from fibers of polyolefins and polyesters. Where the fibers used are incapable of absorbing liquids, they may be treated with a surfactant for improved wettability. The material selected for the cover may be porous to allow rapid passage of liquid. An example of one suitable material is heat bonded or point bonded nonwoven material comprising polypropylene fibers.

Other materials, which may contain other types of nonwoven fibers, may be used for cover 210. They may include for example a through-air bonded/carded web, a spun-bond bi-component nonwoven web, and a web of cross-linked cellulosic fibers, apertured 3D film or the like. One particular suitable material is available from PGI Nonwovens, Landisville, N.J., and has an overall basis weight of about 40 gsm, with high denier (about 10 denier) bi-component fibers situated on the top and low denier (about 6 denier) bi-component fibers situated on the bottom. The bi-component fibers are optionally made of a polypropylene inner core and polyethylene outer sheath. Preferably, the material used should be nonabsorbent and should permit the passage of liquid, but it may include hydrophilic fibers such as pulp within the interstices of the material.

Another exemplary cover 210 may be formed from a liquid permeable film such as a 3-D apertured poly sheet comprising conical holes, available from Tredegar, located in Richmond, Va. Other substrate materials are contemplated as well.

Cover 210 of absorbent article 200 is intended to be positioned proximal to the user's skin. Cover 210 is liquid permeable, allowing liquid to pass through to the rest of absorbent article 200. Cover 210, if used, is preferably compliant, soft feeling and non-irritating to the user's skin. Cover 210, if used as a topsheet, can be made from any of the materials conventional for this type of use, for example spunbonded polypropylene or polyethylene, polyester, RAYON, Hydrofil® nylon fiber available from Allied Fibers, or the like. One suitable material is a hydrophilic 15 gsm spunbond polypropylene nonwoven from Avgol Nonwoven Industries, located in Holon, Israel. Another is a 17 gsm wettable nonwoven coverstock, made of thermal bond polypropylene, available from PGI Nonwovens, Landisville, N.J.

Other non-limiting examples of suitable materials that can be used as cover 210, in its use as a topsheet, are woven and nonwoven polyester, polypropylene, polyethylene, NYLON, and RAYON and formed thermoplastic films. Suitable films are described, for example, in U.S. Pat. No. 4,324,246 to Mullane and Smith and U.S. Pat. No. 4,342,314 to Radel and Thompson, both of which patents are incorporated herein by reference. Formed films may be selected for cover 210 when used as a topsheet because they are permeable to liquids and yet non-absorbent. Thus, the surface of the formed film, which is in contact with the body, remains substantially dry and is more comfortable to the wearer.

Cover 210 may be adhesively secured in place by any suitable construction adhesive or hydrophilic adhesive, such as cycloflex adhesive available from National Starch and Chemical, Bridgewater, N.J.

Barrier layer 240 is positioned on the opposite side of absorbent layer 230. Barrier layer 240 is the portion of the absorbent article 200 that is distal from the user's skin. Barrier layer 240 is preferably a liquid impermeable material such as a poly blend. Barrier layer 240 is proximal to, or in some embodiments attached to, clothing such as an undergarment in use. Barrier 240 blocks the passage of any unabsorbed liquid from article 200 and provides support for the absorbent layer. Exemplary features of the absorbent layer 230, which includes two elongated portions 232 and 233, will be described hereinafter in greater detail.

Materials suitable for use in forming barrier 240, which is configured to prevent the passage of liquid, are well known in the industry. Such materials include, for example, films such as polyethylene, polypropylene, and copolymers, as are known in the absorbent article art. Suitable materials may include for example a liquid-impermeable laminate comprising a soft nonwoven (cloth-like/hydrophobic) on the outside and fluid-impermeable film (low gauge poly) on the inside. An example of this is a poly laminate available from Clopay Plastic Products Company, Cincinnati, Ohio, which consists of 0.6 mil polyethylene film and 17 gsm (gram per square meter) SMS (spunbond/meltblown/spunbond) nonwoven. Another version is a poly laminate 9B-396 available from Pliant Corporation of Newport News, Va., which consists of 0.3 mil copolymer film and 14 gsm SBPP (spunbond polypropylene) nonwoven. However, other laminate variations may be used in various gauges and basis weights. For instance, other polymers (polypropylene, olefins, polyester, co-extruded polymers, etc.) or coatings (adhesive, synthetic rubber, latex, polyurethane, etc.) can be used in place of the polyethylene film. Other material components (polypropylene, polyethylene, bi-component fibers, polyester, cotton, RAYON, NYLON, olefins, etc.) can be used in either woven or nonwoven (spunbond, thermal bond, through-air bond, etc.) construction in place of the SMS outer cover. The preferred fluid-impermeable film for the liquid-impermeable laminate is a breathable 0.8 mil polyethylene version, which contains calcium carbonate, available from Tredegar Film Products, Richmond, Va. This material allows water vapor to pass through it, but does not permit the liquid itself to pass through it.

Absorbent article 200 also includes absorbent layer 230 having separate components forming elongated portions 232 and 233, which may be at least two strips of absorbent material, laterally spaced from one another. Elongated portions 232 and 233 define the boundary of elongated gap 234. Absorbent layer 230 may be formed from an absorbent material such as an airlaid. Further, the airlaid material may include Super Absorbent Polymer (SAP), in which the SAP optionally has a basis weight in the range of about 300 to about 500 gsm. Still further, the airlaid may have a SAP concentration of about 50% by weight.

The term SAP as used herein encompasses a hydrocolloid material, which is capable of absorbing many times its own weight of aqueous liquid. These materials are generally prepared by polymerizing one or more monomers, which if homopolymerized by conventional methods, would form water-soluble polymers. To render them water insoluble, these polymers or mixtures of them are typically crosslinked. Known polymers of this type are based on cross-linked salts of polyacrylic acid or polymethacrylic acid. Exemplary superabsorbent materials suitable for use include polyacrylamides, polyvinyl alcohol, ethylene maleic anhydride, and the like. Preferred are SAP's comprising crosslinked salts of polyacrylic acid.

The SAP may have a relatively uniform particle size, or may have a distribution of particle sizes. An exemplary form of SAP is a granular or powdered material having a distribution of particle sizes ranging from about 45 μm to about 850 μm, preferably between about 106 μm and about 850 μm. The presence of some proportion of particles of small size may encourage effective penetration of such particles into the absorbent layer 230, and may also increase the rate of liquid uptake when the absorbent article receives a liquid insult, due to the high surface area per unit weight of small particles.

The absorbent layer 230, made up of elongated portions 232 and 233, is interposed between the cover 210 and the barrier layer 240. The absorbent layer 230 is configured to absorb an initial insult at an initial absorbency rate and to absorb a subsequent insult at a subsequent absorbency rate faster than the initial absorbency rate.

The elongated portions 232 and 233 of the absorbent layer 230 may be secured in contacting relation to the barrier layer. The barrier layer 240 can be maintained in contact with the absorbent layer elongated portions 232 and 233 by applying adhesive, optionally in spaced, limited areas, to an inner surface of the barrier layer 240. Additionally, cover 210 may be adhered to barrier layer 240 by the application of an adhesive material at a location corresponding to the elongated gap 234.

Examples of suitable adhesives used for this purpose include the acrylic emulsion E-1833BT manufactured by Rohm and Haas Company of Philadelphia, Pa. and acrylic emulsions manufactured by H. B. Fuller Company of St. Paul, Minn. Additionally, water-absorbing adhesives may be used, such as are known in the art. Also contemplated are thermoplastic hot melt adhesives such as 34-563A, available from National Starch, Inc.

In accord with one exemplary aspect of the present invention, as a liquid insult is introduced to the absorbent article, the liquid insult passes through a liquid permeable cover to the absorbent layer. As the liquid is absorbed by the absorbent layer, the absorbent layer begins to expand. According to an exemplary embodiment of the present invention, the liquid collects in the gap defined by the absorbent layer and the elongated portions of the absorbent layer absorb most of the liquid from the side walls adjacent the gap.

As the elongated portions of the absorbent layer absorb the liquid, these portions expand upwardly creating a deeper gap, and increasing the open area through which the liquid can move farther into the absorbent layer. As the elongated portions absorb more liquid, they begin to become gel-blocked. The result of this gel-blocking effect is that the fluid in the gap or a channel defined by the absorbent layer will migrate to the areas of the elongated portions farther away from the point of insult to unused regions of the absorbent layer. This ultimately leads to faster, more effective absorption.

Referring specifically to FIG. 2A, that figure illustrates one embodiment, including cover 210, barrier layer 240, and absorbent layer 230. The absorbent layer 230 includes elongated portions 232 and 233 which are shown in FIG. 2A in a dry, pre-absorption state. Elongated portions 232 and 233 are configured to expand after an introduction of an insult, thereby urging separation of cover 210 from barrier layer 240, as shown in FIG. 2B. After subsequent insults, elongated portions 232 and 233 of absorbent layer 230 are urged to further separate cover 210 from barrier layer 240. The expansion of elongated portions 232 and 233 is continued after a plurality of insults until a maximum absorption of absorbent layer 230 is reached.

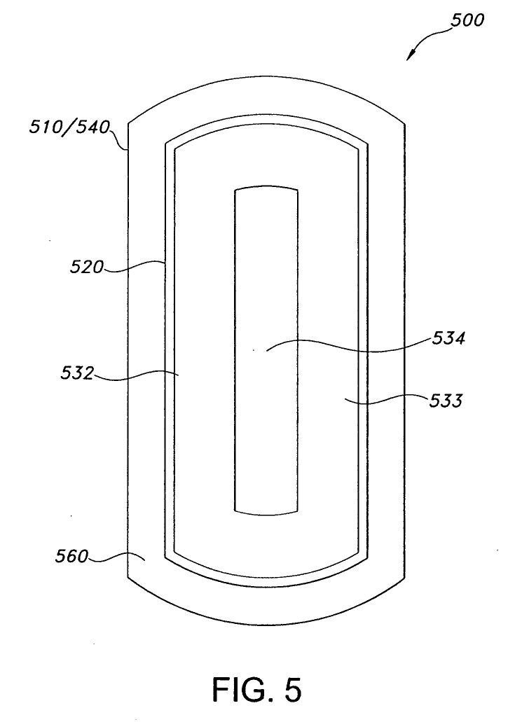

In another embodiment of the present invention, as exemplified in FIG. 3, the absorbent article may also be provided with a transfer layer 320. In this embodiment, a cover 310 is configured and positioned to permit the passage of liquid to the transfer layer 320, thereby utilizing cover 310 as a topsheet. Thus, in this embodiment, transfer layer 320 is interposed between the cover/topsheet 310 and a barrier layer 340. Transfer layer 320 may be formed from a material such an airlaid, wherein the airlaid may further include material such as SAP. The SAP airlaid used to provide a transfer layer may have a lower density such as in the range of about 150 gsm to about 200 gsm, or other effective ranges.

More specifically, FIG. 3A illustrates an embodiment of an absorbent article including a cover 310, a transfer layer 320, a barrier layer 340, and an absorbent layer 330. The absorbent layer 330 includes elongated portions 332 and 333 which are shown in FIG. 3A in a dry, pre-absorption state. Elongated portions 332 and 333 are configured to expand after an introduction of an insult, thereby urging separation of cover 310 from barrier layer 340, as shown in FIG. 3B. After subsequent insults, elongated portions 332 and 333 of absorbent layer 330 are urged to further separate cover 310 from barrier layer 340. The expansion of elongated portions 332 and 333 is continued after a plurality of insults until a maximum absorption of absorbent layer 330 is reached.

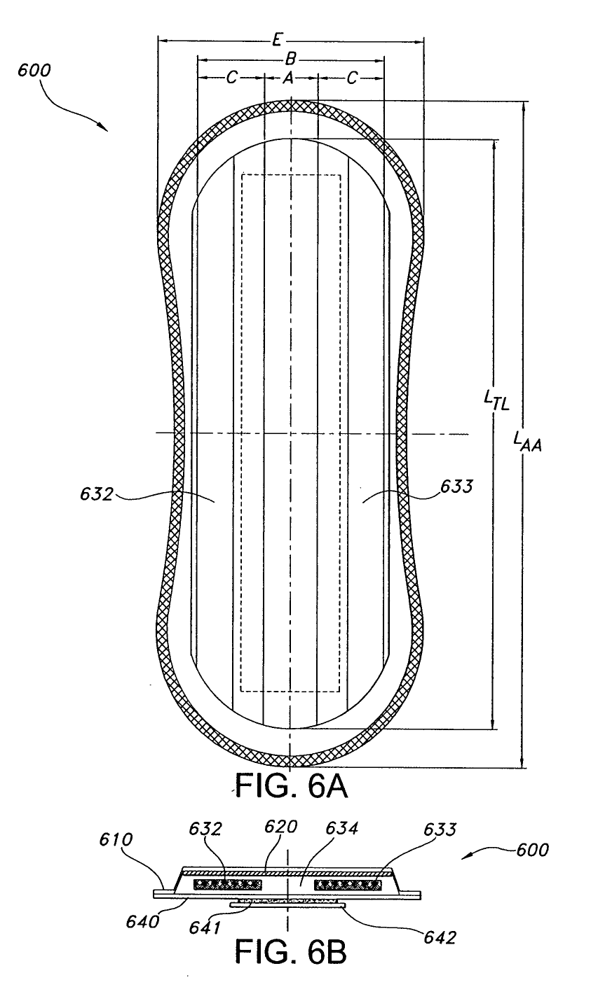

As illustrated in FIG. 4, a pair of elongated strips 432 and 433 of an absorbent layer are separated by a channel 434 formed in the absorbent layer. Channel 434 extends for at least a portion of a length of the article, such as the length of a transfer layer 420, wherein the elongated portions 432 and 433 are substantially parallel to one another and are not connected at the two opposing ends of each portion 432 and 433. The absorbent article shown in FIG. 4 also includes a cover or topsheet 410 and a barrier layer 440. The absorbent article also includes a perimeter region 460 at which the topsheet 410 and barrier layer 440 are optionally bonded.

The absorbent article embodiment illustrated in FIG. 4 is substantially rectangular in shape. Specifically, the illustrated embodiment includes a rectangular topsheet 410 and a rectangular barrier layer 440, a rectangular transfer layer 420, and rectangular absorbent layer portions 432 and 433.

Though a rectangular configuration may optionally be selected, other shapes are contemplated as well, depending on the size of the absorbent article, the intended use for the absorbent article, and other design considerations. Also, the configurations of the respective components of the absorbent article may differ from one another. For example, though an outer perimeter of the article may be substantially rectangular, the transfer layer, absorbent layer, and other components may have rounded shapes or different configurations. Further, the portions of the absorbent layer that define the channel or gap are optionally provided with the same or different shapes or sizes, depending on specific design criteria.

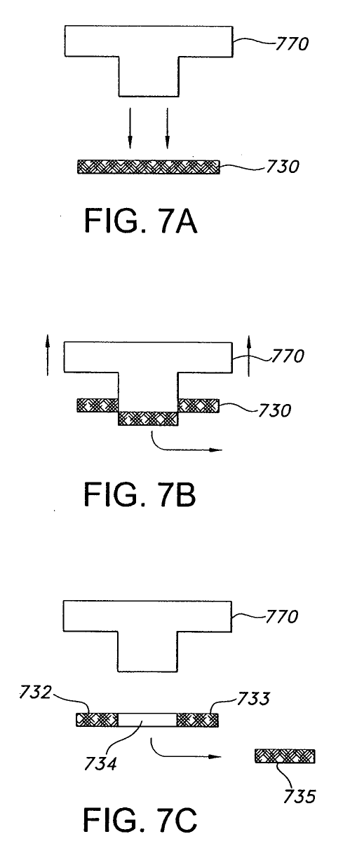

In another embodiment, as shown in FIG. 5, the absorbent article 500 has an absorbent layer that includes elongated portions 532 and 533 in which the elongated portions are substantially parallel with each other along at least a portion of the length of the article and form a perimeter region at least partially surrounding a gap 534.

More specifically, FIG. 5 illustrates an embodiment of an absorbent article 500 including a cover 510, a transfer layer 520, a barrier layer 540, and an absorbent layer having elongated portions 532 and 533. The cover 510 and barrier layer 540 are joined or attached or adhered along a perimeter region 560. As in other embodiments, elongated portions 532 and 533 are configured to expand after an introduction of an insult, thereby urging separation of cover 510 from barrier layer 540.

As illustrated in both FIGS. 4 and 5, the elongated gap is exemplified by having the feature of being longer than it is wide. Nevertheless, other configurations are contemplated as well. For example, one or more gaps formed by the absorbent layer may be elongated in a direction transverse or angled with respect to the length of the absorbent article. Also, more than two absorbent layer portions are optionally utilized in order to provide plural gaps or channels.

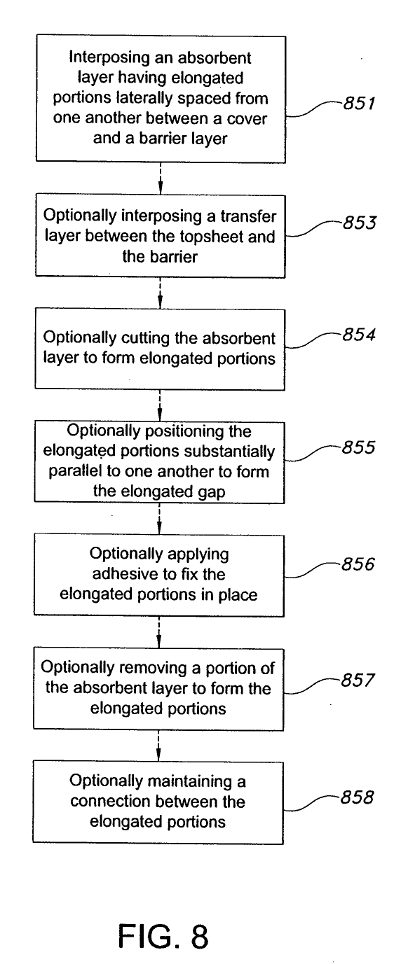

FIG. 6A is a top view illustration of another embodiment of an absorbent article according to an aspect of the invention. This embodiment, generally designated by the numeral 600, is in the form of a feminine hygiene pad and is intended to be used within an undergarment of a user.

More specifically, FIG. 6A illustrates an absorbent article 600 including a cover, a transfer layer, a barrier layer, and an absorbent layer having elongated portions 632 and 633. The cover and barrier layer are joined or attached or adhered along a perimeter region. As in other embodiments, elongated portions 632 and 633 are configured to expand after an introduction of an insult, thereby urging separation of the cover from the barrier layer.

FIG. 6B is a side view illustration of the embodiment of FIG. 6A. Referring specifically to FIG. 6B, absorbent article 600 includes a cover 610, a transfer layer 620, a barrier layer 640, and an absorbent layer having elongated portions 632 and 633. The elongated portions 632 and 633 together define a gap 634. The cover 610 and barrier layer 640 are joined or attached or adhered along a perimeter region. The article also has a positioning adhesive layer 641 to secure the absorbent article 600 in place, such as to an undergarment of the wearer of the absorbent article. The adhesive layer is covered with releasable paper 642.

As shown in FIG. 6A, absorbent article 600 has an overall length defined by L_{AA} and a transfer layer length defined by L_{TL}. In addition, absorbent article 600 also has an absorbent article overall width E and an overall absorbent layer width B. An average transfer layer width is slightly wider than the overall absorbent layer width B. Also shown in FIGS. 6A and 6B, elongated portions 632 and 633 of the absorbent layer each have a width C, defining a gap of width A.

Recognizing that a wide variety of shapes and dimensions can be selected for components of an absorbent article according to this invention, and without being limited to any dimensions or proportions, the following exemplary dimensions are optionally selected for the absorbent article 600 shown in FIGS. 6A and 6B:

| | |
|---|---|
| L_{AA} | 245 mm |
| L_{TL} | 217 mm |
| A | 20 mm |
| B | 70 mm |
| C | 25 mm |
| E | 100 mm |

Accordingly, and according to one exemplary embodiment of the invention, the gap 634 defined by the absorbent layer is optionally about 20% to about 35% of the overall width of the absorbent layer and more preferably about 25% to 30% of the overall width of the absorbent layer. Also, the width of gap 634 is between about 70% and about 90% of the width of each elongated portion 633 and 632, more preferably about 75% and about 85% of the width of each elongated portion 633 and 632, and most preferably about 80% of the width of each elongated portion 633 and 632.

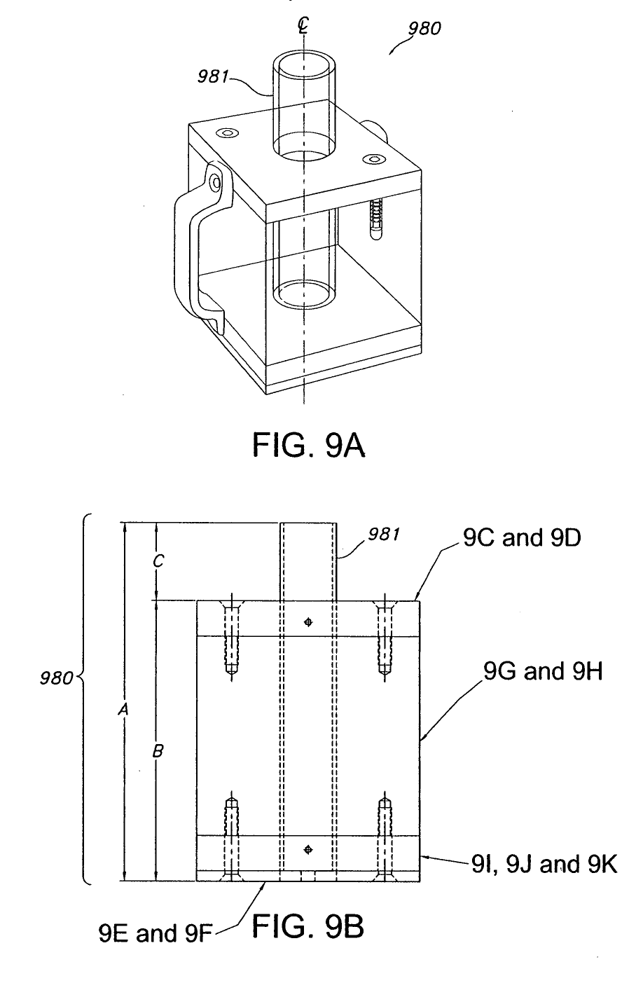

The present invention also provides a method of manufacturing the absorbent layer. Specifically, included is the method of manufacturing the elongated portions of the absorbent layer. One embodiment of such a method is illustrated in FIGS. 7A, 7B, and 7C. A cutting tool 770, which can be any cutting tool known in the art, such as a blade or a die or a punch, is applied to a sheet of absorbent material 730 suitable for use as an absorbent layer. As cutting tool 770 is applied against absorbent material 730, a gap is created by the removal of section 735 from the remainder of the absorbent material 730. Cutting tool 770 is separated from absorbent material 730 creating gap 734, defined by the remaining elongated portions 732 and 733.

The method illustrated in FIG. 7 is especially suited for the preparation of an absorbent layer, such the absorbent layer defining elongated portions 532 and 533 in FIG. 5, that has a continuous outer region that substantially or completely surrounds an inner gap. For such use, the cutting tool 770 may be shaped to define the inner surface of the absorbent layer that defines the interior gap.

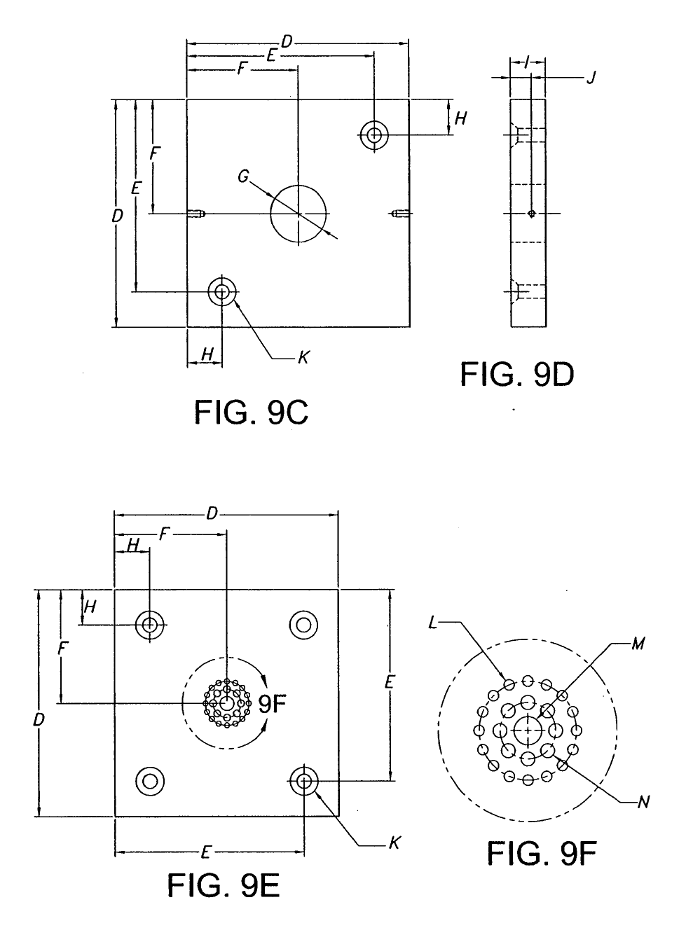

The present invention also provides a method of making the absorbent article. FIG. 8 illustrates by flow diagram the method of making the absorbent article. The method of making the absorbent article comprises the step, 851, of interposing an absorbent layer having elongated portions laterally spaced from one another between a cover and a barrier layer, thereby defining an elongated gap between the laterally spaced portions of the absorbent layer. The absorbent article may include a SAP-impregnated material or a pulp-containing layer (optionally containing SAP), and/or a layer or layers designed to enhance fluid distribution in and across the absorbent layer, proximal the side of the absorbent layer nearest the cover. Thus, the liquid that comes into contact with the cover seeps through the cover and comes into contact with the absorbent layer and is absorbed. The absorbent layer swells upon absorption and forms a gel. The barrier layer inhibits the passage of any unabsorbed liquid through the article, thereby preventing accidental wetting or soiling of the wearer's clothing.

FIG. 8 also lists additional, optional steps which may also be included in the method of making the absorbent article. Step 853 includes the additional, optional step of interposing a transfer layer between the cover, acting as a topsheet, and the barrier layer. Step 854 includes the additional, optional step of cutting the absorbent layer to form elongated portions, as is shown in more detail in FIGS. 7A, 7B and 7C. Step 855 includes the additional, optional step of positioning the elongated portions of the absorbent layer substantially parallel to one another to form the elongated gap. Step 856 includes the additional, optional step of applying adhesive to fix the elongated portions in place. Step 857 includes the additional, optional step of removing a portion of the absorbent layer to form the elongated portions. Step 858 includes the additional, optional step of maintaining a connection between the elongated portions.

One of the advantages of the present invention is that the absorbent article has an absorption rate that, after the first insult, is faster. This is accomplished without compromising rewet characteristics.

Rewetting, which occurs when an absorbent material becomes saturated with liquid, is the transmission of fluid back through the absorbent article cover, and results in a “rewetting” of the cover and, ultimately, discomfort to the wearer. As more fluid is absorbed and the absorbent layer becomes increasingly saturated, liquid therefore has a tendency to permeate back through the topsheet resulting in increased discomfort to the wearer. Thus, it is advantageous to provide an absorbent article having a faster absorbency rate without causing increased, undesirable rewetting effects.

According to an exemplary embodiment of the invention, the absorbent article is characterized by a first absorbency rate associated with a first insult and a second absorbency rate associated with a second insult, where the second absorbency rate is faster than the first absorbency rate. In other words, the rate of insult absorption for the second insult is faster than that for the first insult of equal size. Even after a third insult occurs, the rate of absorbency further increases or decreases less than about 10%. This is again accomplished without a significant compromise to rewet characteristics. The advantage of the increased absorbency is that discomfort to the wearer is shorter because the liquid is absorbed much faster. This is accomplished without significantly changing the absorbent capacity of the product, which would result in discomfort.

In many cases, in order to increase the absorbency rate, products can optionally be provided with additional absorbent layers, embossing, channeling, or using absorbant layers with higher densities and higher concentrations of absorbent materials. The present invention, according to one exemplary embodiment, avoids the need for increasing the amount of absorbent material used, and instead requires less absorbant material to accomplish improved performance.

Exemplary features of the invention are illustrated in the following examples.

### EXAMPLE 1

Tests were conducted using an absorbent product design in which two 20 mm strips of SAP airlaid were laid 30 mm apart to form an absorbent layer. Using 15 articles, or pads, 3 absorption rates were measured using an absorbency rate tester. Each insult had a volume of 30 ml. The following results were produced:

| Description | | | | | | |
|---|---|---|---|---|---|---|
| of Product | Time (s) | | | Rewet (gram) | | |
| Pads | 1^{st} | 2^{nd} | 3^{rd} | 1^{st} | 2^{nd} | 3^{rd} |
| 1 | 20.12 | 4.07 | 4.13 | 0.08 | 11.95 | 15.80 |
| 2 | 18.63 | 3.29 | 3.62 | 0.10 | 12.00 | 15.96 |
| 3 | 19.75 | 3.47 | 3.34 | 0.07 | 13.08 | 15.52 |
| 4 | 18.84 | 3.07 | 3.41 | 0.07 | 12.11 | 15.18 |
| 5 | 23.50 | 3.05 | 4.37 | 0.05 | 12.43 | 14.40 |
| 6 | 19.15 | 4.12 | 4.03 | 0.09 | 8.00 | 16.06 |
| 7 | 17.69 | 3.12 | 3.41 | 0.07 | 9.22 | 15.95 |
| 8 | 19.16 | 3.53 | 3.43 | 0.10 | 12.06 | 15.78 |
| 9 | 19.28 | 3.38 | 3.84 | 0.09 | 11.56 | 15.53 |
| 10 | 20.91 | 3.84 | 3.13 | 0.07 | 13.03 | 15.91 |
| 11 | 20.59 | 3.28 | 4.22 | 0.26 | 13.55 | 16.19 |
| 12 | 19.47 | 4.69 | 4.67 | 0.12 | 11.54 | 16.02 |
| 13 | 19.75 | 3.97 | 4.41 | 0.21 | 13.47 | 16.40 |
| 14 | 20.10 | 3.53 | 4.72 | 0.24 | 14.81 | 16.31 |
| 15 | 19.69 | 4.16 | 4.22 | 0.12 | 13.33 | 16.12 |
| Average= | 19.78 | 3.64 | 3.93 | 0.12 | 12.14 | 15.81 |
| St. dev.= | 1.30 | 0.48 | 0.51 | 0.01 | 1.70 | 0.50 |

As is shown from the foregoing data, the average absorption rate of the 15 samples was 19.78 seconds for the first insult. For the second insult, the average absorption rate dropped considerably, and unexpectedly. Specifically, the average for the 15 samples for the second insult was 3.64 seconds. Surprisingly, the third insult also retained a considerably low rate of absorption. The average rate of absorption for the 15 samples for the third insult was 3.93 seconds, or less than about 10% greater than the second insult. The average rewet results after the first, second, and third insults were 0.12 gram, 12.14 gram, and 15.81 gram, respectively.

### EXAMPLE 2

Tests were conducted using an absorbent product design in which two 25 mm strips of SAP airlaid were laid 20 mm apart to form an absorbent layer. Using 15 articles, 3 absorption rates were measured using an absorbency rate tester. Each insult had a volume of 30 ml. The following results were produced:

| Description | | | | | | |
|---|---|---|---|---|---|---|
| of Product | Time (s) | | | Rewet (gram) | | |
| Pads | 1^{st} | 2^{nd} | 3^{rd} | 1^{st} | 2^{nd} | 3^{rd} |
| 1 | 21.34 | 5.56 | 4.03 | 0.09 | 6.86 | 15.99 |
| 2 | 20.56 | 4.34 | 4.19 | 0.07 | 4.04 | 16.07 |
| 3 | 20.75 | 4.31 | 4.10 | 0.08 | 7.15 | 15.68 |
| 4 | 19.91 | 4.44 | 4.72 | 0.08 | 5.46 | 15.83 |
| 5 | 21.31 | 4.63 | 4.84 | 0.07 | 6.64 | 15.82 |
| 6 | 21.03 | 4.06 | 4.16 | 0.07 | 5.43 | 15.58 |
| 7 | 21.97 | 4.40 | 4.68 | 0.07 | 5.22 | 15.88 |
| 8 | 18.31 | 5.47 | 4.35 | 2.72 | 6.98 | 15.77 |
| 9 | 21.81 | 4.56 | 3.66 | 0.07 | 5.58 | 16.09 |
| 10 | 20.81 | 4.57 | 5.41 | 0.06 | 5.34 | 15.92 |
| 11 | 20.59 | 4.12 | 3.65 | 0.07 | 5.89 | 15.39 |
| 12 | 20.81 | 4.62 | 4.03 | 0.07 | 11.76 | 15.52 |
| 13 | 20.57 | 4.25 | 3.72 | 0.07 | 10.42 | 15.38 |
| 14 | 21.43 | 4.97 | 3.78 | 0.05 | 9.05 | 15.11 |
| 15 | 21.15 | 4.41 | 4.09 | 0.09 | 8.92 | 15.09 |
| Average= | 20.82 | 4.58 | 4.23 | 0.25 | 6.98 | 15.67 |
| St. dev.= | 0.87 | 0.44 | 0.50 | 0.68 | 2.16 | 0.32 |

As is shown from the data, the average absorption rate of the 15 samples was 20.82 seconds for the first insult. For the second insult, the average absorption rate once again dropped considerably, and unexpectedly. The average for the 15 samples for the second insult was 4.58 seconds. And even more surprising, the third insult not only retained a considerably low rate of absorption, but the rate of absorption was 4.23 seconds, less than the average rate for the second insult. The rewet results after the first, second, and third insults were 0.25 gram, 6.98 gram, and 15.67 gram, respectively.

### EXAMPLE 3

For purposes of comparison, tests were conducted using absorbent articles having a monolithic absorbent layer as opposed to the elongated portions provided according to one embodiment of this invention. Each insult had a volume of 30 ml. The test data is reproduced below:

| Description | | | | | | |
|---|---|---|---|---|---|---|
| of Product | Time (s) | | | Rewet (gram) | | |
| Pads | 1^{st} | 2^{nd} | 3^{rd} | 1^{st} | 2^{nd} | 3^{rd} |
| 1 | 30.09 | 31.17 | 38.14 | 0.05 | 7.48 | 13.22 |
| 2 | 46.99 | 63.95 | 128.12 | 0.05 | 12.15 | 16.24 |
| 3 | 48.39 | 72.17 | 131.83 | 0.06 | 9.07 | 15.89 |
| 4 | 54.53 | 66.64 | 134.95 | 0.05 | 8.56 | 16.37 |
| 5 | 49.75 | 63.02 | 125.09 | 0.06 | 9.02 | 15.96 |
| 6 | 47.27 | 66.33 | 127.00 | 0.04 | 8.90 | 16.04 |
| 7 | 46.12 | 60.11 | 169.63 | 0.06 | 9.19 | 16.18 |
| 8 | 42.63 | 52.01 | 157.70 | 0.02 | 11.20 | 16.34 |
| 9 | 47.54 | 70.13 | 138.35 | 0.05 | 9.24 | 16.47 |
| 10 | 40.57 | 74.19 | 131.52 | 0.06 | 9.74 | 16.12 |
| Average= | 45.39 | 61.97 | 128.23 | 0.05 | 9.46 | 15.88 |
| St. dev.= | 6.56 | 12.55 | 34.77 | 0.01 | 1.33 | 0.95 |

The test results show that for the second insult, the absorption rate, as measured using an absorbency rate tester, is slower requiring, on average, an absorption time approximately 37% longer. The decrease in the absorption rate was even greater for the third insult. Tests showed an average increase of over 200% more time for liquid absorption.

More specifically, test results for the first insult using 10 samples showed an absorption rate of 45.39. For the second absorption rate, the average of 10 samples for the second insult was 61.97, or approximately 37% longer. The average absorption rate for the third insult was 128.23, or over 200% longer. The rewet results after the first, second, and third insults were 0.05 gram, 9.46 gram, and 15.88 gram, respectively.

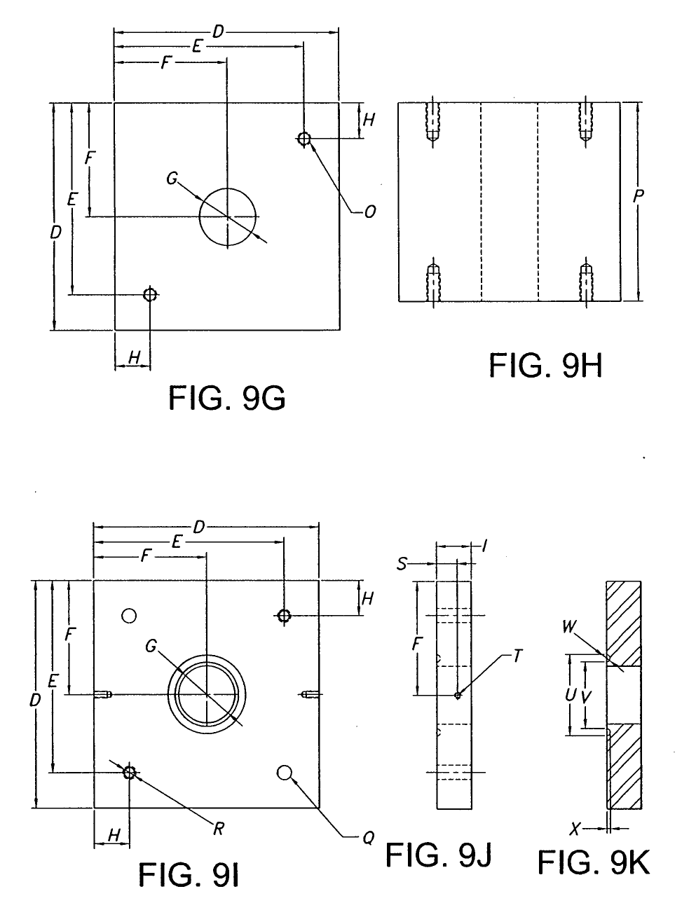

Referring now to FIGS. 9A through 9K, details of the absorbency rate tester, generally designated by the numeral (980), will now be described. Referring to FIG. 9A, the tester, which is essentially a modified strike-through plate, includes a body and a clear tube 981 through which a liquid is introduced. As is shown in FIG. 9B, the tester includes a top plate component (shown in FIGS. 9C and 9D), a body (shown in FIGS. 9G and 9H), another plate component (shown in FIGS. in 9I, 9J and 9K), and a base plate component (shown in FIGS. 9E and 9F). The tube 981 is formed from tubing material having an inside diameter of 0.875 inch and an outside diameter of 1 inch. The tube 981 has a length of 6 ⅛ inch and it is clear. The total weight of the tester shown in FIG. 9A, including the handles, is 3549.00 grams. The weight of the handles alone is 87.50 grams.

The following table lists the dimensions of the components illustrated in FIGS. 9A-9K:

| Dimension | Measurement (Inch) |
|---|---|
| A | 6.313 |
| B | 4.938 |
| C | 1.375 |
| D | 4.000 |
| E | 3.375 |
| F | 2.000 |
| G | 1.000 Ream Thru, 1-Place |
| H | 0.625 |
| I | 0.625 |
| J | 0.367 |
| K | Drill & C'Sink For M6 F.H.M.S., 2-Places |
| L | 0.094 Dia. Thru 16-Places Eq. Sp. On a 0.875 Dia. B.C. |
| M | 0.250 Dia. Thru, 1-Place |
| N | 0.125 Dia. Thru, 8-Places Eq. Sp. On a 0.500 Dia. B.C. |
| O | M6 × 1.00 Pitch Tap × .500 D.P., 2-Places, Both Ends |
| P | 3.500 |
| Q | Tapped M6 − 1.00 Thru (2 Places) |
| R | 6 mm Dia. Thru, 2-Places |
| S | 0.258 |
| T | M3 × 0.50 Pitch × 10 mm DP. Both Sides |
| U | 1.422 Dia. |
| V | 1.167 Dia. |
| W | .125 Dia. |
| X | 0.063 |

Referring to FIGS. 9E and 9F, the bottom plate of the tester is provided with a central region with apertures for the flow of fluid from the tube 981 to an absorbent garment (not shown) below the tester. The plate is formed from 3/16 inch thick by 4 inch by 4 inch LEXAN. The total weight of the bottom plate component shown in FIGS. 9E and 9F and the plate component shown in FIGS. 9I-9K, described below, is 1233.60 grams total.

Referring now to FIGS. 9G and 9H, the body component of the tester is formed from 3.50 inch thick by 4 inch by 4 inch clear polyurethane. The weight of the body component is 1019.20 grams.

Referring now to FIGS. 9I-9K, the plate component shown in those figures has a surface defining an o-ring groove so as to provide a liquid barrier between the upper surface of the lower plate shown in FIGS. 9E and 9F and the lower surface of the plate shown in FIGS. 9I-9K. The o-ring detail is specifically shown in FIG. 9K. The plate shown in FIGS. 9I-9K is formed from ⅝ inch thick by 4 inch by 4 inch 303 stainless steel. The o-ring that is used with the plate is provided by McMaster-Carr under part number AS568A216.

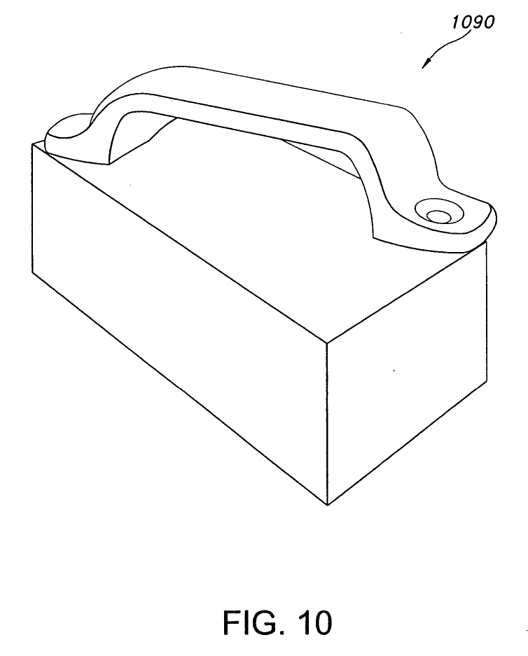

For completing the tests set forth above, the following apparatuses and materials were required: (1) Balance; (2) 1 liter storage container with lid; (3) 5 liter plastic pitcher; (4) spatula or non-metallic spoon; (5) magnetic stirrer and magnetic stirring bar; (6) hot plate for heating 500 ml of de-ionized H₂O; (7) plastic weighing trays; (8) hot and cold de-ionized H₂O; (9) iodine free NaCl; (10) certified food color, green shade #15794; (11) a Burette clamp; (12) a 125 ml separatory funnel; (13) a ring stand or equivalent; (14) a large beaker or bottle, at least 100 ml; (15) a modified strike-through plate (absorbency rate tester) 4″×4″ weight−7.8 lbs. (980; FIGS. 9A and 9B); (16) 30 ml, 1% Saline; (17) a timer or stopwatch; (18) a #617 Ahlstrom filter papers, 2″×4″ (md×cd); (19) a stanley knife or scissors; and (20) a 4.4 lb rectangular weight (2″×4″) weight=0.5 psi. (1090; FIG. 10).

The tests were conducted using the following procedures:
1) Prepare 1% saline solution (with concentrated dye additive):
   a). Prepare concentrated dye solution.
      1) Place a dry 1000 ml plastic jar onto the balance and tare.
      2) Weigh 20 g of dye powder into the 1000 ml plastic jar.
      3) Place the magnetic stirring bar into the jar.
      4) Place the jar with the dye and stirrer onto the magnetic stirrer.
      5) Add approximately 500 ml of hot de-ionized H₂O into the jar.
      6) Turn on the stirrer at a slow speed and stir for about a half hour's time.
      7) After the half hour's time, add 500 ml of room temperature de-ionized H₂O to top up the jar for a total of 1000 ml. Place a lid on the jar and continue to stir for another half hour to complete dissolution of the powdered dye.
      8) Use this concentrated dye in step b). for coloring the saline solution used in product testing.
   b). Prepare 1% saline solution.
      1) Place 5 liter plastic pitcher onto the balance and tare.
      2) Add 4,950.0 g of de-ionized H₂O into the 5 liter pitcher then remove from the balance.
      3) In a plastic weighing dish, weigh out 50.0 g NaCl.
      4) Add the 50 g of NaCl to the pitcher of deionized H₂O and stir with the plastic spoon until the NaCl is thoroughly dissolved.
   c). Add approximately 10 ml of the concentrated dye made in step a) to the 1% saline solution made in step b). (Adjust the desired color shade by adding more or less dye concentrate.)
2) Tape absorbent article onto table with tape and stretch to make flat.
3) Place absorbency rate tester (FIG. 9A) over center of product at the predicted insult area.
4) Slide separatory funnel over center of tube of absorbency rate tester absorbent article, so that hole in absorbency rate tester (980; FIGS. 9A and 9B) is centered (Cₜ; FIG. 9A) under funnel tip.
5) Make sure stopcock on separatory funnel is closed and stopwatch is zeroed.
6) Dispense 30 ml solution from plastic beaker into the separatory funnel.
7) Start the stopwatch and simultaneously dispense the fluid into absorbency rate tester (980; FIGS. 9A and 9B). Take care to always open the stopcock in the same direction.
8) Close stopcock.
9) Watch through transparent cylinder (981; FIGS. 9A and 9B) and at the surface of the product until fluid flows past absorbency rate tester and is no longer present on the surface of the product.
10) Record result to the nearest 0.01 seconds.
11) Remove absorbency rate tester (980; FIGS. 9A and 9B) and let product sit for 10 minutes.
12) Weigh 10 filter papers and record weight on filter papers.
13) After 10 minutes, place weighed filter papers and the 4.4 lb. weight (1090; FIG. 10) in center of insult area. Let weight remain for 2 minutes.
14) Remove weight and filter papers. Reweigh filter papers and subtract dry weight of filter papers to calculate rewet.
15) Repeat steps 3) through 14) two more times, for a total of three insults.
16) Calculate: Wet filter paper(g)−dry filter paper (g)=Rewet (g).
17) Report: Absorbency rate (s) and Rewet (g).

While preferred embodiments of the invention have been shown and described herein, it will be understood that such embodiments are provided by way of example only. For example, absorbent articles according to the invention may be used in a variety of absorbent articles, including for example diapers, adult incontinence pads, and feminine hygiene products. Numerous variations, changes and substitutions will occur to those skilled in the art without departing from the spirit of the invention. Accordingly, it is intended that the appended claims cover all such variations as fall within the spirit and scope of the invention.

What is claimed:

1. An absorbent article comprising:
a barrier layer configured to prevent the passage of liquid;
a cover; and
an absorbent layer interposed between said cover and said barrier layer, said absorbent layer being configured to absorb an initial insult at an initial absorbency rate and to absorb a subsequent insult at a subsequent absorbency rate faster than said initial absorbency rate.

2. The absorbent article of claim 1, said absorbent layer having elongated portions laterally spaced from one another, thereby defining an elongated gap between said laterally spaced portions of said absorbent layer.

3. The absorbent article of claim 1 wherein said cover provides a topsheet.

4. The absorbent article of claim 1 wherein said cover provides a transfer layer adjacent said cover.

5. The absorbent article of claim 4 wherein said cover is configured and positioned to permit the passage of liquid to said transfer layer.

6. The absorbent article of claim 1 further comprising a transfer layer.

7. The absorbent article of claim 1 wherein said absorbent layer comprises at least two separate components forming said elongated portions.

8. The absorbent article of claim 1 wherein said absorbent layer comprises at least two strips of absorbent material forming said elongated portions.

9. The absorbent article of claim 2 wherein said elongated portions together define a channel.

10. The absorbent article of claim 9 wherein said absorbent layer comprises a perimeter region at least partially surrounding said channel.

11. The absorbent article of claim 1 wherein said absorbent layer comprises a perimeter region that completely surrounds said elongated gap.

12. The absorbent article of claim 1 wherein said cover and said barrier layer are joined or attached or adhered along a perimeter region.

13. The absorbent article of claim 2 wherein said elongated gap defined by said elongated portions of said absorbent layer is longer than it is wide.

14. The absorbent article of claim 1 wherein said absorbent layer is fixed in place with respect to said barrier layer by an adhesive.

15. The absorbent article of claim 14 wherein said adhesive is applied to said barrier layer at a location corresponding to said gap.

16. The absorbent article of claim 14 wherein said adhesive is not applied to said barrier layer at a location corresponding to said gap.

17. The absorbent article of claim 2 wherein said elongated portions of said absorbent layer are configured to expand after an insult, thereby urging separation of said cover from said barrier layer.

18. The absorbent article of claim 17 wherein said elongated portions of said absorbent layer are configured to expand after a subsequent insult, thereby urging further separation of said cover from said barrier layer.

19. The absorbent article of claim 17 wherein said elongated portions of said absorbent layer are configured to expand after plural insults until a maximum absorption of said absorbent layer is reached.

20. The absorbent article of claim 1, wherein the absorbent layer is configured to absorb a second subsequent insult at a third absorbency rate that is greater than said second absorbency rate.

21. The absorbent article of claim 20, wherein said third absorbency rate is less than about 10% greater than said second absorbency rate.

22. The absorbent article of claim 1, wherein the absorbent layer is configured to absorb a second subsequent insult at a third absorbency rate that is less than said second absorbency rate.
